(19) United States
(12) Patent Application Publication
(10) Pub. No.: US 2003/0130249 A1
(43) Pub. Date: Jul. 10, 2003

(54) Therapeutic use of estrogen receptor (ER)beta-selective agonists for triggering somatotropic, organotropic and anticatabolic effects (somatotropic therapy)

(75) Inventors: Walter Elger, Berlin (DE); Gudrun Reddersen, Jena (DE); Birgitt Schneider, Jena (DE); Alexander Hillisch, Jena (DE); Olaf Peters, Jena (DE); Dirk Kosemund, Erfurt (DE); Gerd Muller, Jena (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/272,290

(22) Filed: Oct. 17, 2002

Related U.S. Application Data
(60) Provisional application No. 60/331,529, filed on Nov. 19, 2001.

(30) Foreign Application Priority Data
Oct. 17, 2001 (DE) 101 51363.1

(51) Int. Cl. A61K 31/56

(52) U.S. Cl. 514/182

(57) ABSTRACT

This invention describes the use of estrogen receptor (ER)β-selective agonists for the production of a pharmaceutical agent for triggering somatotropic and/or organotropic effects in the CNS, the circulatory system, the skeletal system and/or the immune system in the aging male and female organism (anticatabolic therapy).

This application claims the benefit of the filing date of U.S. Provisional Application Serial No. 60/331,529 filed Nov. 19, 2001.

This invention relates to the use of estrogen receptor (ER)β-selective agonists for the production of a pharmaceutical agent for triggering somatotropic and/or organotropic effects in the CNS (central nervous system), in the muscles, in the circulatory system, the skeletal system and/or the immune system in the aging male and female organism.

### INTRODUCTION

### Role of Estrogens for Sexual Dimorphisms

Estrogens play a central role for sexually dimorphous functions and physical traits. Androgens of the fetal testes dominate the somatic sexual differentiation [1]. In humans, a testicular hormone secretion can be detected during the embryonal development starting from the seventh week [2-4]. In the fetal rat, the fetal testosterone secretion begins around the 15th day of embryonal development [5]. This testosterone secretion results in the stabilization of the wolffian ducts, from which the male gonaducts develop and lead to the development of male accessory sexual glands and male external sex organs. In this early phase of ontogenesis, ovarian hormones (estrogens) do not seem to play any morphogenetic role in the two sexes. The elimination or absence of testicular androgens is sufficient by itself to produce a female development of the genital tract and external sex organs (basic femaleness) [1].

Estrogens can induce sexual dimorphisms even in later life. These functions do not relate only to the female sex. They play a deep-seated role also outside of the reproduction functions and relate to all important organ systems, especially also the heart and blood vessels, the CNS, the locomotor system, the immune system, important endocrine glands, such as the pancreas, the liver, and the skin. Corresponding dimorphisms are expressed by, for example, differences in the growth processes before and during sexual maturity [6]. In adulthood, they manifest themselves in, i.a., different body masses and compositions, differences in protein, carbohydrate, fat and bone metabolism, as well as in differences in circulatory functions and in the immune system. To some extent, diseases of these systems often occur very differently between the two sexes. Examples of this are the significantly higher circulatory morbidity rate and mortality of the male sex and the higher risk of osteoporosis of the female sex. The female sex is also more greatly affected by auto-immune diseases and degenerative diseases of the central nervous system.

Sexually dimorphous functions take place in part by irreversible imprinting of morphological and functional features. Corresponding sex differences persist even in the absence of sex hormones all one's life. Sexual dimorphisms result in part by modulation of functions by circulating estrogens or estrogens that are generated in the tissue. In this connection, there are numerous examples. Such functions are often modulated by other sex hormones, e.g., androgens [6, 7].

### Role of Gonadal and Peripheral Hormone Secretion

It is obvious that estrogens secreted via the ovary in the female sex play an important role. In addition, estrogens can be formed from precursors in many organs, and tissues can be formed from testosterone and adrenal androgens, such as androstenedione, and over several steps from dehydroepiandrosterone [8]. The latter mechanisms apply for both sexes. In the male sex, the peripheral formation of estrogens by far outweighs that of the gonads.

With the onset of menopause, the female organism can also generate estrogens only by the metabolic conversion of adrenal steroids, while the latter is possible in the male sex from gonadal (testosterone) and adrenal precursors. The secretion of testosterone in the male sex that is obtained at least partially at later ages should be one of the reasons why estrogen deficits are less dramatic and manifest themselves later in males.

With this limitation, it can also be assumed with men that with the drop both in gonadal and in adrenal secretion of the precursors of estrogen formation [9, 10], estrogen deficiency conditions occur with the results that are known in the female sex, such as osteoporosis and feelings of ill health. Corresponding deficiency conditions can also occur in the wake of therapeutic measures, for example by the suppression of the adrenal secretion of DHEA and androstenedione by large-dose glucocorticoids [9].

Estrogens that are formed in the target tissue are involved decisively in the (fetal) sexual differentiation of the central nervous system [11]. A significant functional importance of estrogens formed in the various regions of the CNS in terms of paracrine and autocrine functions is also to be assumed in all phases of postnatal life.

The presence of the enzyme that catalyzes the formation of estrogens, the aromatase, can be detected in many tissues and organs, except for in the CNS, for example in the bony tissue [12, 13]. Earlier [14], organs that were successful for estrogens (for example uterus and vagina) were distinguished from organs that were not regarded as organs that were successful for estrogens. The variably high content of estrogen receptors in the tissues and thus the differing abilities of the organ to accumulate and retain radiolabeled estradiol were decisive for this overhauled classification system. This ability was very pronounced for organs such as the uterus and vagina but not, however, for others, for example the liver. In the meantime, endocrinological studies and modem molecular biological methods have demonstrated the expression of estrogen receptors and their functions in almost all organs and tissues [12].

### Molecular Aspects of Estrogen Action

After the discovery that estrogens are specifically bonded in the cell, the idea prevailed for several decades that there is only one estrogen receptor. It has now become certain that several such receptors exist, the estradiols bind specifically with high affinity and control the expression of genes as transcription factors that are controlled by ligands. In addition, it was found that estrogen receptors not only act on the DNA with their “individual” binding sites; rather they can interact with other receptor proteins and their transcription factors in a complex way.

In addition to the long-known “standard” estrogen receptor (now estrogen receptor alpha/(“ERα”)), a second receptor, the estrogen receptor beta (“ERβ”) [15-19] was discovered and examined with respect to its function. In the meantime, some tests have been published that have as their object the variable function of both estrogen receptors. Their distribution in the organism is variable. Organs with a preponderance of “ERα” are, i.a., the uterus, vagina, mammary gland, liver and hypophysis. “ERβ” dominates, i.a., the ovary, the prostate, the circulatory system and individual nuclei of the CNS. Most organs express both estrogen receptors (ER alpha/ER beta) [17].

Essential findings on the function of ERα and ERβ result from observations of genetically altered mice, in which in each case, one of the two estrogen receptors or ERα and ERβ were excluded [17]. Almost all functions that are known from estrogens are no longer present with ERα. The failure of ERα results in the two sexes in the loss of reproductiveness. A loss of ERβ has few dramatic results. Female animals still have a cycle but are subfertile. Other authors pursued the question of whether ERα and ERβ represent mutually modulating systems analogously to the physiology of the andreno receptors [18]. These authors discovered considerable changes of the estrogen receptor (ERα) expression in different sexually dimorphous core centers of the CNS in the case of ERβ knock-out mice. The results of the ERβ loss also affected the expression of the progesterone receptor. The data that were taken are an indication that the ERβ affects the imprinting of neural structures and functions in the phase of sexual differentiation in the two sexes and also controls the expression of ERα and the pattern of reactions in an estrogen treatment in later life. These observations are of special theoretical importance, since according to previous ideas, estrogens do not play any role in the organization of the female CNS (sexual differentiation). Obviously, however, the ERβ also plays an important role with the female sex in this ontogenesis phase.

### Therapeutic Importance of Estrogens

Estrogens are used in oral contraceptives in combination with a gestagen to avoid undesired pregnancies. In addition to ovulation, the endogenous hormone secretion is suppressed by a corresponding hormone treatment. A largely normal menstrual pattern is maintained by the hormonal active ingredients that are supplied. The metabolic functions of the suppressed ovarian hormones are also substituted by the active ingredients that are supplied. In hormonal contraceptive agents, ethinylestradiol or mestranol—a prodrug of ethinylestradiol—are the only estrogens used. In this context, an essential action of ethinylestradiol is its strong inhibitory action on the secretion of FSH. This is important to suppress the maturation of a follicle in the treatment cycle. This anti-gonadotropic activity of the ethinylestradiol is enhanced by the simultaneous administration of a gestagen and is supplemented by an inhibition of the LH secretion. The strong estrogenic effects of ethinylestradiol in the liver [20-22] are a problem in the use of ethinylestradiol. Said effects result in changes of a broad spectrum of metabolic effects, i.a., in changes of bile secretion [20], the renin-angiotensin-aldosterone system [23], the hepatic hemostasis factors and the lipoproteins [21]. These changes are presumably the bases of side effects that can accompany the use of hormonal contraceptives.

After the ovarian hormone secretion runs out in post-menopause, estrogens are used as “estrogen (hormone) replacement therapy” (ERT or HRT). This therapy raises deficiency symptoms that manifest themselves especially in feelings of ill-health, circulation functions and an increased degeneration of the bone substance. In this therapy, the standard estrogen effects on the uterus and the mammary glands are undesirable. The growth processes in the mucous membrane of the uterus that are triggered by estrogens require the simultaneous use of gestagens, since otherwise the risk of suffering from a carcinoma of the endometrium increases [24]. This measure, however, is not possible without the occurrence of drawbacks elsewhere, since gestagens in the breast, unlike in the uterus, do not inhibit the proliferation in this organ [25, 26]. There therefore exist ideas that the HRT in combination with a gestagen results in an increased risk of suffering from a breast carcinoma. Just like the use of oral contraceptives, the oral ERT or HRT results in deviations of a broad spectrum of liver functions and also results in a measurable increase of the risk of undergoing deep vein thromboses and the associated complications. The latter problem could also not be remedied by the use of SERMs (raloxifene, tamoxifen), since these substances are not anti-estrogenic in the liver, but rather act as estrogens. A significantly increased risk of diseases in the wake of clotting disorders accompanies tamoxifen and raloxifene [27, 28].

### THE OBJECT OF THIS INVENTION

The object of this invention is to make available a therapy that preserves and enhances the central aspects of a hormone therapy with female sex hormones with the emphasis on advantageous metabolic effects, without being burdened by their negative aspects.

Induction of atrophy of the testes, reduced testosterone level in the blood in men

Actions of the estrogen on the endometrium in women

Actions of estrogen and gestagen on the mammary glands

Direct estrogen effects on the liver functions.

The special feature of the invention is also confirmed in that it can be used in the male sex without serious drawbacks. A stimulation of the testosterone secretion is achieved by the substances according to the invention in addition to a spectrum of advantageous organotropic and metabolic actions. Conventional estrogens inhibit the testicular secretion of testosterone.

This object is achieved by the use according to the invention of estrogen receptor (ER)β-selective agonists for the production of a pharmaceutical agent for triggering somatotropic and/or organotropic effects in the CNS, in the muscles, in the circulatory system, the skeletal system and the immune system in the aging male and female organism.

In addition to the above-described use of the ERβ-selective agonists, this invention also relates to a corresponding “method-of-treatment” for the cited indications with ERβ-selective agonists.

The essence of the therapy is in the regeneration of mechanisms that in puberty trigger the development of organ functions outside of the genital tract and manifest themselves as a catabolic process with the lessening of the gonad function in both sexes. The purpose of the therapy is to counteract the degeneration of the muscles and bone substance that takes place within the context of aging and the reduction of important organ functions: anticatabolic therapy.

In the therapy according to the invention, the activation of the GH-(growth hormone) IGF-I axis plays an important role in the same manner as a stimulation of the adrenal and testicular androgen secretion.

In addition to aging-induced and therapeutically-induced disorders of the metabolism, the proposed therapy can advantageously influence, for example, the catabolic effects of a therapy with glucocorticoids.

The invention is based on the finding that estrogen receptor (ER)β-selective agonists, surprisingly enough, exert effects on somatotropic and organotropic functions:

Stimulation of the growth hormone and IGF-I, which in all organ systems exerts advantageous effects on their growth and development

Growth and development of muscle mass

Growth and development of bone mass

Growth and development of the thymus and its function

The substances according to the invention and increased IGF-I levels have an advantageous effect on the age-related degeneration of neurons

Stimulation of the adrenal secretion of androgenic hormones (DHEA, DHEA-S, androstenedione). The latter are metabolized into estrogens and stronger androgens in the tissue and contribute to advantageous effects in the tissue.

Stimulation of the testicular hormone secretion (testosterone): advantageous metabolic effects, positive effects on ill-health and libido.

Increased IGF-I levels in the blood result in increased burning of fat; the uptake of glucose in the tissue is promoted.

Lipoproteins: The increase of HDL-cholesterol reduces the retention of cholesterol in the vessel wall and thus prevents the advance of arteriosclerosis,

which are distinguished quantitatively and qualitatively from those of estradiol and take place in a dose range that is (far) below the dose range in which the corresponding ERβ-selective agonist exerts “standard” estrogen effects on the uterus, vagina, gonadotropin secretion and the liver.

The ERβ-selective compounds must thus be dissociated from their abilities to be able to trigger somatotropic and organotropic effects, on the one hand, and “standard” estrogen effects, on the other hand.

The ERβ-selective compounds are used in a dosage in which they trigger virtually no “standard” estrogen effects.

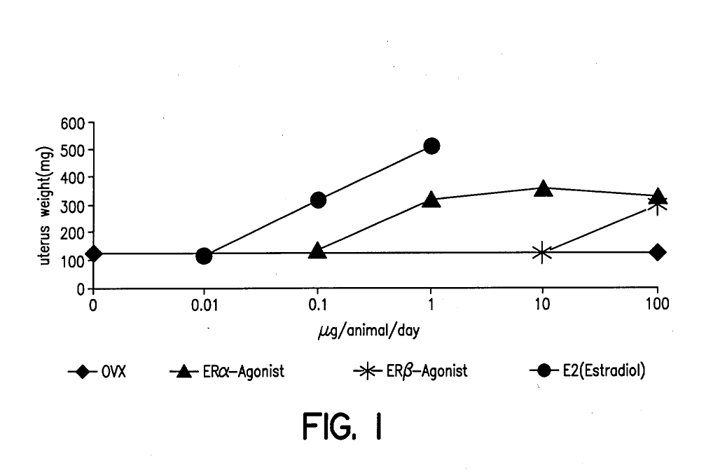

The substances that are to be used according to the invention have very little action in terms of “standard estrogens.” Corresponding estrogenic properties can be examined in the rats that have undergone ovariectomy. In the case of parenteral administration, 17β-estradiol even at a dose of 0.1 μg results in an increase in uterus weight. An ERβ-selective substance that is to be used according to the invention has a comparable uterotropic effect only at a 1000-fold higher dosage (see FIG. 1). These substances have a correspondingly small action on the parameters of hepatic estrogeneity, for example the increase of the angiotensinogen in the blood.

ERβ-selective properties can be studied in the case of female and male rats that are not sexually mature.

Standard estrogen effects can be detected in this model based on the inhibition of the testicular growth and the reduction of the prostate weight. In the case of a parenteral dose of 1 μg/animal/day, estradiol results in a complete inhibition of testicular growth. An ERβ-selective substance according to the invention has a comparable inhibiting effect only at or starting from a 1000-fold higher dosage.

Standard estrogens (estradiol) and an ERα-selective substance have a positive effect on the growth only at very low dosages. Higher dosages inhibit the growth. An ERβ-selective substance that is to be used according to the invention stimulates growth in the same dose range as estradiol but does not have a comparable inhibiting effect in the case of higher dosages.

An ERβ-selective substance that is to be used according to the invention stimulates the secretion of IGF-I more strongly than conventional estrogens.

An ERβ-selective substance that is to be used according to the invention stimulates the testicular growth and the growth of the prostate. This is a more reliable indication of the induction of the secretion of testosterone. Estradiol does not have corresponding stimulating actions on testicular functions in any dose range.

An ERβ-selective substance that is to be used according to the invention stimulates the organ growth of adrenal glands and thymus. Estradiol and an ERα-selective substance have, however, an inhibiting effect on the thymus. The latter is an indication of the induction of a glucocorticoid secretion by conventional estrogens. An ERβ-selective substance that is to be used according to the invention does not have a corresponding disadvantageous effect in any dose range.

An ERβ-selective substance that is to be used according to the invention exerts all therapy-relevant effects in a dose range that lies 10- to 1000-fold below, in which direct effects on the uterus or inhibiting effects on the growing testes are seen.

### ADVANTAGES/PROPERTIES OF THE THERAPY ACCORDING TO THE INVENTION

Estradiol accumulates ERα and ERβ and produces an entire panorama of different effects; the therapy that is proposed here is selective; it has virtually no negative side effects.

It allows the treatment of (senile) atropic conditions, preferably in late and very late ages.

The removal of all catabolic metabolic conditions is possible with it; it results in an improvement of the state of nutrition.

A positive influencing of the metabolic functions (cholesterol) is accomplished with it; for example, a selective increase of the HDL level.

A hormone balance whose patterns are disrupted by age or disease can be corrected.

The androgen secretion of the adrenal gland can be further activated.

In particular, the IGF-1 levels in the blood and in the liver of humans of late or very late age are increased by the treatment according to the invention: the increase of the IGF-1 level has an advantageous influence on all organs.

The influencing of the somatotropic functions (“Everything that makes us fit!”) achieves a better dynamic substance preservation (“Reestablishment of Gender-Dimorphic Metabolic Functions”).

It is quite decisive that under the therapy according to the invention of atropic conditions in late and very late ages, several actions can be achieved simultaneously, namely positive actions on the muscle and bone mass and all organ functions, which are especially affected by natural age degeneration, whereby the stimulation of the GH-/IGF-I axis, the adrenal androgen secretion and, in the male sex, the increased secretion of testosterone play an important role.

This is of great advantage specifically for a therapy that is intended for humans in late or very late ages, since in this phase of life of a human, the functions of several organs are often impaired.

Before and during sexual maturation, various adaptations in the growing organism take place, which have nothing to do with the sexual functions. It was found that by treatment in a prepubescent stage of development with an ERβ-selective agonist, these non-sex organ-related effects can be triggered.

In the course of the natural aging of a human, it is specifically these organ functions that have been positively developed in the youth syndrome that deteriorates.

This invention teaches that these bodily and organ functions that developed in a positive manner in the youth and that deteriorate in the course of the aging process and then result in deficiency symptoms and images of disease, can be restored at least to a certain extent in humans in late and very late ages.

In this connection, examples are the loss of muscle mass and bone mass and the loss of IGF-I, the gonadal and adrenal androgen secretion involved therewith (secretion of DHEA, DHEA-S, androstenedione). Against the background of medical progress in other areas that is helping humans to live longer and longer, this is a very important point. The invention achieves a significant contribution for the quality of life of aging humans, since failures of organs and bodily functions, which mean an impairment of the quality of life, can be corrected or at least mitigated by the invention. The proportion of individuals in the older age brackets in need of care can thus be reduced.

In both older men and women, the use of ERβ-selective agonists according to the invention allows the treatment of catabolic conditions in the wake of a reduced secretion of sex hormones.

In addition, it makes possible the treatment of such catabolic conditions that are caused by a deficiency of the growth hormone and/or IGF.

The secretion of growth hormones and gonadotropic hormones is stimulated by the treatment with an ERβ-selective agonist according to the invention.

The secretion of the IGF-I of the liver and the blood level of this somatotropic factor are increased.

Organotropic effects on the muscles, bones and the CNS are exerted by the increased release of the growth hormone and by its mediators.

In the use of ERβ-selective agonists according to the invention, no direct estrogenic effects are exerted on the sex organs—uterus, vagina, mammary glands—at those dosages that are sufficient for a treatment of catabolic conditions.

Therefore, in the proposed use of an ERβ agonist, the latter usually must not be combined with a gestagen. Indirect estrogen effects, for example by induction of an LH- and FSH-secretion in women, seem possible, but not in post-menopause, since reactive follicles in the ovary are no longer present in this phase.

In addition, considerably reduced effects on estrogen-regulated functions of the liver are observed.

The use of ERβ-selective agonists proposed according to the invention is not an estrogen replacement therapy in the usual sense. In the proposed use, a deficiency of estrogens is not only eliminated, but rather the operability of the affected organs is restored, as is found in younger humans, whereby the reproduction functions that are no longer relevant in the later age (effects on the uterus, mammary gland) are no longer influenced, unlike with conventional estrogens.

Also, under the treatment according to the invention of catabolic conditions with an ERβ-selective agonist in comparison to conventional oral estrogen therapy, which results in a disadvantageous reduction of IGF-I, somatotopic functions are not negatively influenced, and the glucose tolerance is not reduced. Advantageous effects on the lipoprotein pattern in the blood are expected from the therapy according to the invention. The treatment accompanies an increase of the IGF-I blood level and the cardiovascularly protective (“good”) HDL-cholesterol.

The basal endocrine gonad function as well as the thymus function and the immune system are positively influenced.

An ERβ-selective agonist for use for this invention is distinguished by higher affinity to the estrogen receptor of rat prostates in comparison to the rat uterus, or by higher affinity to ERβ in comparison to ERα. This comprises substances that were described in earlier patent applications: “ERβ-Affine Ent-Steroids (WO 00/63228); 16-OH-Steroids (WO 00/47603); Nor-Steroids (WO 01/32680); 8-β-Substituted Steroids (WO 01/77139).” This application also comprises other estrogens that are selective for ERβ that were described in various patent applications or publications, e.g.:

a) ASTRA, Novel Estrogens, WO97/08188, 9502921-1, PCT/SE96/01028;

b) Sumitomo Chemical Co. Ltd., JP 11292872;

c) Androstenediol and Prodrugs of Androstenediol; Pharmaceutical Compositions and Uses for Androstene 3β,17β-Diol, WO99/63973;

d) Phytoestrogens with higher affinity to ERβ in comparison to ERα, such as, for example, the genistein.

The above list is not final.

The ERβ-agonist is preferably selected from 3,16-dihydroxyestra-1,3,5(10)-triene derivatives, (e.g., 3,16α-dihydroxyestra-1,3,5(10)-triene), 8α-H, 9β-H, 10α-H, 13α-H, 14β-H-gonane derivatives, preferably derived from ent-13-alkylgonane (for example ent-estradiol), 8β-substituted estra-1,3,5(10)-triene derivatives and gona-1,3,5(10)-triene derivatives. Examples of preferred ERβ-antagonists are described in DE 199 06 159.9 (WO 00/47603), DE 199 17 930.1 (WO 00/63228), DE 199 41 105.1 and DE 100 19 167.3 (WO 01/77139). Reference is made expressly to the disclosure of these documents, especially to the general structural formulas and preferred single compounds that are shown there.

The compound 8β-vinyl-1,3,5(10)-estratriene-3,17β-diol (WO 01/77139) is most preferred within the scope of this invention for use in the above-described indications.

The selective estrogen action is achieved on the basis of the variable tissue distribution of ERα and ERβ by the subtype-specific ligands. Substances with a preference for ERβ compared with ERα in the in-vitro receptor binding test were described by Kuiper et al. [29].

The pharmaceutical preparations for the use of an ERβ-selective agonist according to the invention contain the latter optionally mixed with pharmacologically common vehicles, adjuvants or diluents as well as optionally with other pharmacologically or pharmaceutically active substances. The production of the pharmaceutical agents is carried out in a known way.

As vehicles and adjuvants, e.g., those are suitable that are recommended or indicated in the following bibliographic references as adjuvants for pharmaceutics, cosmetics and related fields:

Ullmanns Enzyklopädie der technischen Chemie [Ullmann's Encyclopedia of Technical Chemistry], Volume 4 (1953), pages 1 to 39;

Journal of Pharmaceutical Sciences, Volume 52 (1963), page 918 ff., issued by Czetsch-Lindenwald, Hilfsstoffe für Pharmazie und angrenzende Gebiete [Adjuvants for Pharmaceutics and Related Fields];

Pharm. Ind., No. 2 (1961), page 72 and ff.: Dr. H. P. Fiedler, Lexikon der Hilfsstoffe für Pharmazie, Kosmetik und angrenzende Gebiete [Lexicon of Adjuvants for Pharmaceutics, Cosmetics and Related Fields], Cantor K G, Aulendorf in Württemberg 1971.

The compounds can be administered orally, buccally or parenterally, for example intraperitoneally, intramuscularly, subcutaneously or percutaneously. The compounds can also be implanted in the tissue.

For oral administration, the active ingredients can be dissolved or suspended in a physiologically compatible diluent. As diluents, very often oils with or without the addition of a solubilizer, a surfactant, a suspending agent or emulsifier are used. Examples of oils that are used are olive oil, peanut oil, cottonseed oil, soybean oil, castor oil and sesame oil.

The compounds can also be used in the form of a depot injection or an implant preparation that can be formulated such that a delayed release of active ingredient is made possible.

Implants can contain, as inert materials, e.g., biodegradable polymers or synthetic silicones such as, e.g., rubber gum. In addition, the active ingredients can be added to, e.g., a patch for percutaneous administration.

For the production of intravaginal systems (e.g., vaginal rings) or intrauterine systems (e.g., pessaries, coils, IUDs, Mirena^{(R)}) that are charged with active ingredients for local administration, various polymers, such as, e.g., silicone polymers, ethylene vinyl acetate, polyethylene or polypropylene, are suitable.

To achieve a better bioavailability of the active ingredient, the compounds can also be formulated as cyclodextrin clathrates. To this end, the compounds are reacted with α-, β- or γ-cyclodextrin or derivatives of the latter (PCT/EP95/02656).

According to the invention, the active ingredients can also be encapsulated with liposomes.

### METHODOLOGY

### Estrogen Receptor Binding Studies

The binding affinity of the selective estrogens (ERβ ligands) was tested in competitive experiments with use of ³H-estradiol as a ligand in estrogen receptor preparations of the rat prostate and the rat uterus. The preparation of the prostate cytosol and the estrogen receptor test with the prostate cytosol was performed as described by Jung-Testas et al. (1981) [30].

The preparation of rat uterus cytosol as well as the receptor test with the ER-containing cytosol was performed in principle as described by Stack and Gorski, 1985 [31] with some modifications as shown in Fuhrmann et al. (1995) [32].

The ERβ ligands that are claimed for use in this industrial-property right have higher binding affinity to the estrogen receptor ERβ from the rat prostate than from the rat uterus (ERα). In this case, it is assumed that ERβ predominates in the rat prostates over ERα, and ERα predominates in the rat uteri over ERβ. In accordance-with this, we find that the ratio of the binding to prostate and uterus receptors is identical qualitatively to the quotient of the relative binding affinity (RBA) to human ERβ and rat ERα (according to Kuiper et al.) [29].

To examine the action of the ERβ-selective agonists in the context according to the invention, their estrogenic action on the genital tract, liver functions, somatotropic factors and on the secretion of gonadotropins was examined in comparison to estradiol in adult rats that have undergone ovariectomies.

The action on the somatotropic functions, gonad functions and the genital tract was studied in infant, gonad-intact, male and female rats.

The dosage of the ERβ-selective agonist in the context of this invention is between 10 μg and 10 mg daily absolute.

The invention is to be explained in more detail by FIGS. 1 to 8.

The ERα-selective agonist that is used is readily 3,17β-dihydroxy-19-nor-17α-pregna-1,3,5(10)-triene-21,16α-lactone (DE 100 48 634.7).

As an ERβ-selective agonist, in each case 8β-vinyl-1,3,5(10)-estratriene-3,17β-diol (DE 100 19 167.3 or WO 01/77139) was used.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are -set forth uncorrected in degrees Celsius, and all parts and percentages are by weight, unless otherwise indicated.

FIG. 1

Determination of the uterotropic action of an ERα- or ERβ-selective agonist (agon) in comparison to estradnol (E₂) Study of adult rats that have undergone ovariectomies 14 days after the ovariectomies, treatment day 1-day 3, autopsy day 4, subcutaneous administration in 0.2 ml of vehicle.

Result: E₂ and ERα-agon induce uterus growth in the case of much lower dosage than the ERβ-agon; 0.1 μg of E₂ and a 1000×higher dose (100 μg) of ERβ-agon have comparable “standard” estrogenic activity.

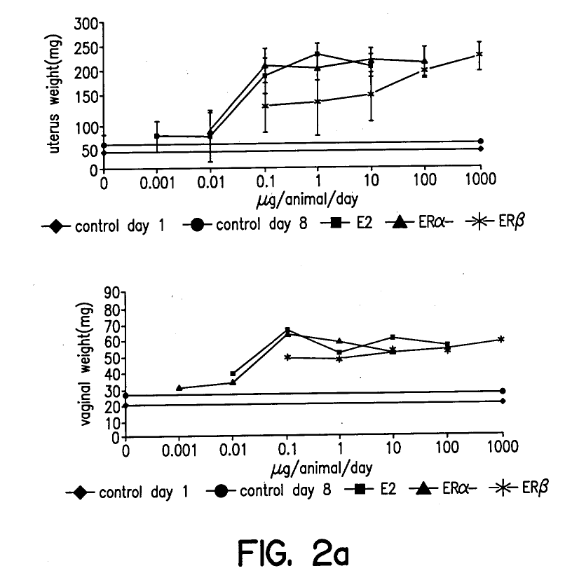

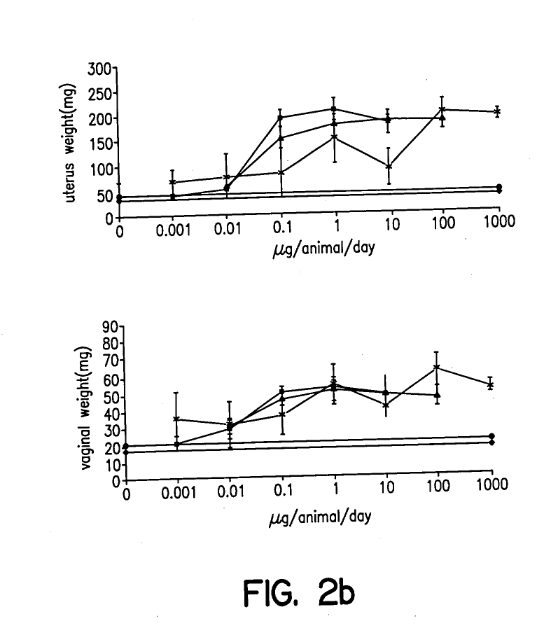

FIG. 2

Effects of ERβ-agon or ERβ-selective agonists (agon) (E₂) on the uterus and vagina in ovary-intact, sexually immature rats in comparison to estradiol. Treatment of the young animals after weaning over 7 days (day 1-day 7, autopsy day 8, subcutaneous injection) in two tests with different dose ranges.

Result: In the presence of ovaries, the ERβ-agon has considerable effects on the weight development of the uterus and vagina in extremely low dosages. In the lowest tested dose, the effects of ERβ-agon exceed those of ERα-agon, and E₂ is statistically significant. This effect is an indication of the induction of ovarian estrogen secretion.

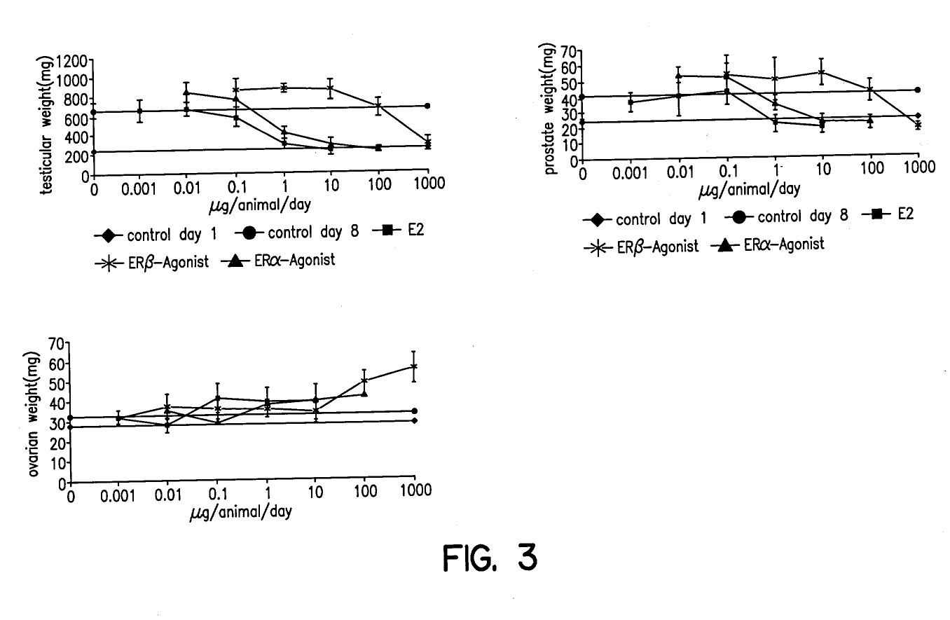

FIG. 3

Effects of ERβ-agon or ERβ-selective agonists (agon) (E₂) on gonads of male and female, sexually immature rats in comparison to estradiol. Treatment of the young animals after weaning over 7 days (day 1-day 7, autopsy day 8, subcutaneous injection).

Result: In the test phase in control animals, the testes show a very quick growth; the ovaries, however, show very little. E₂ inhibits the growth of the testes in a dose-dependent manner. The simultaneous inhibition of the growth of the prostate reflects the suppression of the testosterone secretion by E₂. ERβ-agon stimulates testicular growth beyond the normal size, which is statistically significant. The acceleration of the growth of the prostate confirms that with the stimulation of the testicular growth, an increased secretion of testosterone by ERβ-agon results. The inhibition of the testicular growth that occurs with high doses of ERβ-agon reflects its “standard” estrogeneity.

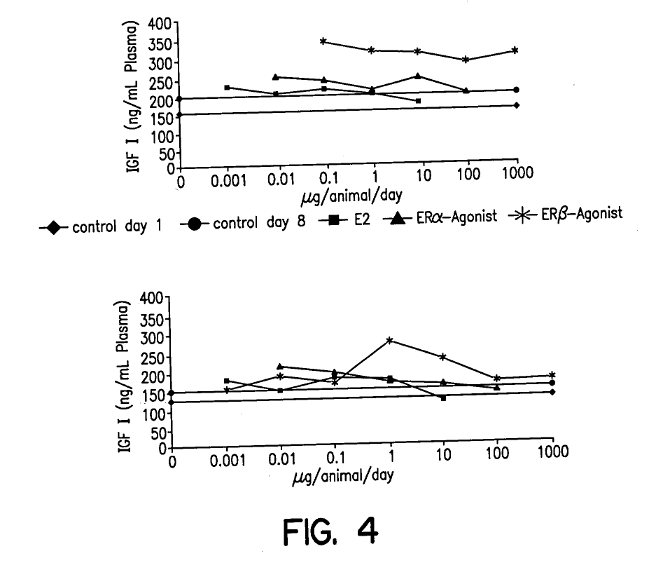

FIG. 4

Effects of ERα- or ERβ-selective agonists (agon) on IGF-I in the plasma from sexually immature rats with intact ovaries in comparison to estradiol (E₂). Treatment of the young animals after weaning over 7 days (day 1-day 7, autopsy day 8; subcutaneous injection) in two tests with different dose ranges.

Result: Statistically significant increase of the IGF-I under ERβ-agon via wide dose ranges. No comparable effects under ERα-agon and E₂ in the tested dose range.

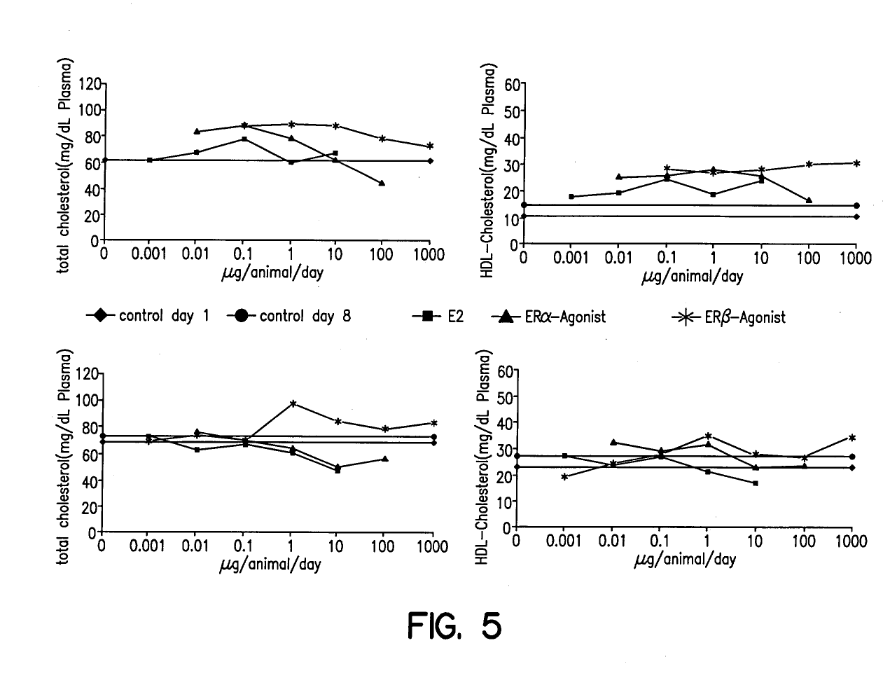

FIG. 5

Effects of ERα- or ERβ-selective agonists (agon) on the cholesterol fractions in the plasma from sexually immature rats with intact ovaries in comparison to estradiol (E₂). Treatment of the young animals after weaning over 7 days (day 1-day 7, autopsy day 8, subcutaneous injection) in two tests with different dose ranges.

Result: In contrast to ERα-agon and E₂, ERβ-agon via the increase of the HDL-cholesterol fraction results in elevated plasma levels of the total cholesterol in a broad dose range. High doses of ERₐ-agon and E₂ drop the HDL-cholesterol fraction and the total cholesterol.

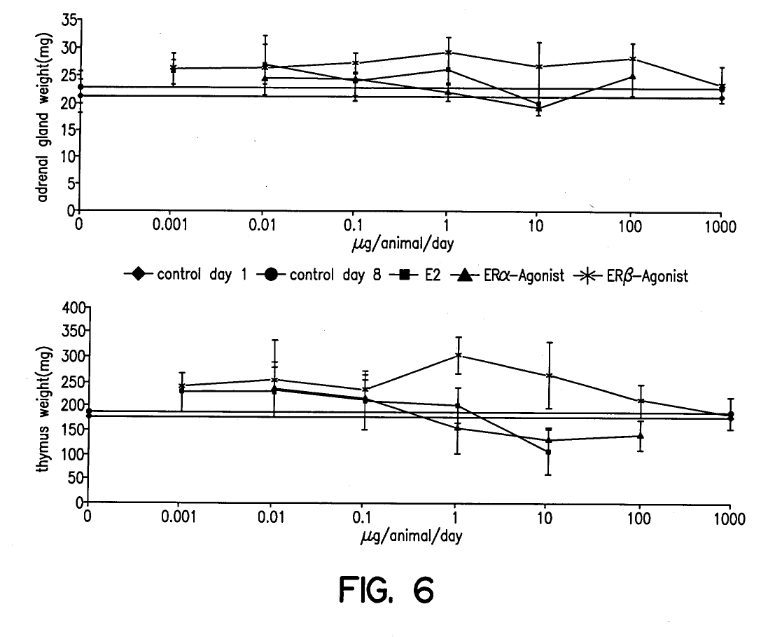

FIG. 6

Effects of ERα- or ERβ-selective agonists (agon) on the development of the organ weights of the adrenal glands and the thymus of sexually immature rats with intact ovaries in comparison to estradiol (E₂). Treatment of young animals after weaning over 7 days (day 1-day, 7, autopsy day 8, subcutaneous injection).

Result: Over a broad dose range, ERβ-agon results simultaneously in an increase of adrenal gland weight and thymus weight. In contrast to this, ERα-agon and E₂ result in less pronounced positive effects and via “standard” estrogeneity result in a reduction of the thymus weight in the case of higher dosages.

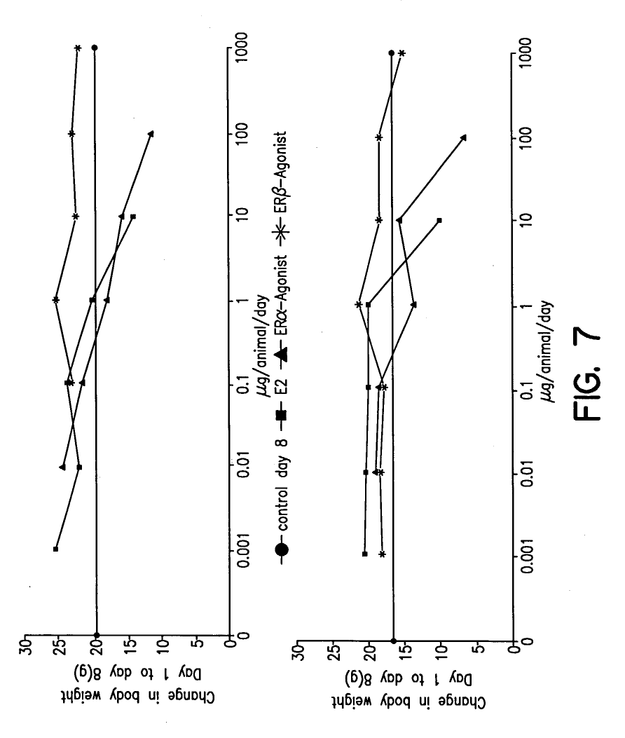

FIG. 7

Effects of ERα- or ERβ-selective agonists (agon) on the body growth of sexually immature rats whose ovaries are intact in comparison to estradiol (E₂). Treatment of the young animals after weaning over 7 days (day 1-day 7, autopsy day 8, subcutaneous injection) in two tests with different dose ranges.

Result: ERβ-agon results in an accelerated growth of the animals via a broad dose range. Effects with lower dosages are more pronounced than with higher dosages. In contrast to this, ERα-agon and E₂ result in a considerable reduction of the weight increase with higher dosages.

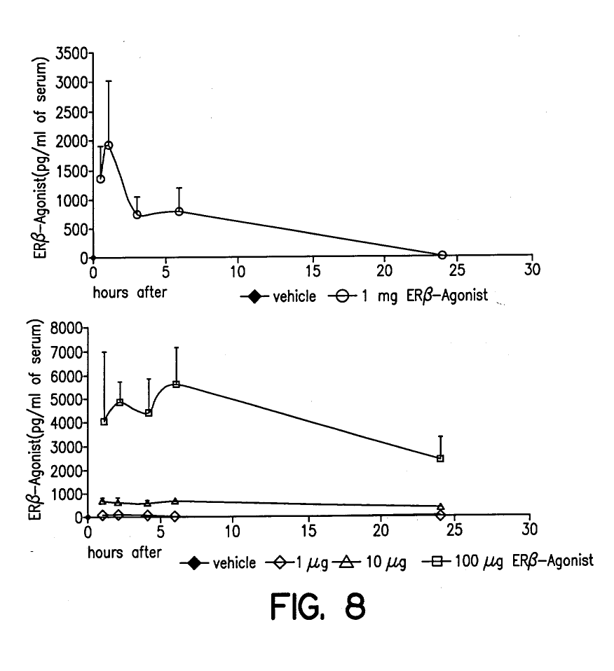

FIG. 8

Plasma levels of the ERβ-selective agonist after a one-time oral or parenteral (subcutaneous injection) administration to ovariectomized rats, determinations 0.5; 1; 3; 6; 24 hours after oral administration of 1.0 mg (left) or 1; 2; 4; 6; 24 hours after subcutaneous administration of graduated dosages. Immunological determination by means of LCMS-validated Radio Immuno Assay (RIA).

Result: The ERβ-agonist that is used here by way of example is orally readily bioavailable.

The entire disclosures of all applications, patents and publications, cited herein and of corresponding German Application No. 101 51 363.1, filed Oct. 17, 2001, and U.S. Provisional Application Serial No. 60/331,529, filed Nov. 19, 2001, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### REFERENCES

[1] Jost, A.

Role des gonades foetales dans la differentiation sexuelle somatique [Role of Fetal Gonads in Somatic Sexual Differentiation].

Arch. Anat. Micr. Morph. Exper. 1947, 36 pp. 271-315

[2] Rey, R.; Picard, J. Y.

Embryology and Endocrinology of Genital Development.

Baillieres Clin Endocrinol Metab (England), April 1998, 12(1), pp. 17-33

[3] Balboni, G. C.; Barni, T.; Gloria, L. et al.

Alfa-inhibin and Transferrin in Human Fetal Testis.

Ital J Anat Embryol (Italy), 1995, 100 Suppl 1, pp. 519-24

[4] Murray, T. J.; Fowler, P. A.; Abramovich, D. R., et al.

Human Fetal Testis: Second Trimester Proliferative and Steroidogenic Capacities.

J Clin Endocrinol Metab (United States), December 2000, 85(12), pp. 4812-7

[5] Majdic, G.; Saunders, P. T.; Teerds, K. J.

Immunoexpression of the steroidogenic enzymes 3-beta hydroxy steroid dehydrogenase and 17 alpha-hydroxylase, C17,20 lyase and the receptor for luteinizing hormone (LH) in the fetal rat testis suggests that the onset of Leydig cell steroid production is independent of LH action.

Biol Reprod (United States), February 1998, 58(2), pp. 520-5

[6] Liu, J. L.; Yakar, S.; Le Roith, D.

Mice deficient in liver production of insulin-like growth factor I display sexual dimorphism in growth hormone-stimulated postnatal growth.

Endocrinology (United States), Dec. 2000, 141(12), pp. 4436-41

[7] Span, J. P.; Pieters, G. F.; Sweep, C. G., et al.

Gender Difference in Insulin-like Growth Factor I Response to Growth Hormone (GH) Treatment in GH-Deficient Adults: Role of Sex Hormone Replacement.

J Clin Endocrinol Metab (United States), March 2000, 85(3), pp. 1121-5

[8] Schweikert, H. U.

[Intersexuality: Gonadal Dysgenesis and Testicular Feminization]

Gynakologe [Gynecologist] (Germany), February 1995, 28(1), pp. 17-26

[9] Arit, W.; Callies, F.; Koehler, I., et al.

Dehydroepiandrosterone Supplementation in Healthy Men with an Age-Related Decline of Dehydroepiandrosterone Secretion.

J Clin Endocrinol Metab United States), October 2001, 86(1.0), pp. 4686-92

[10] Baulieu, E. E.; Thomas, G.; Legrain, S., et al.

Dehydroepiandrosterone (DHEA), DHEA Sulfate, and Aging: Contribution of the DHEAge Study to a Sociobiomedical Issue.

Proc Natl Acad Sci USA (United States), Apr. 11, 2000, 97(8), pp. 4279-84

[11] Naftolin, F.; Ryan, K. J.; Davies, I. J., et al.

The Formation of Estrogens by Central Neuroendocrine Tissues.

Recent Prog Horm Res (United States), 1975, 31, pp. 295-319

[12] Schweikert, H. U.; Wolf, L.; Romalo, G.,

Estrogen Formation from Androstenedione in Human Bone.

Clin Endocrinol (Oxf) (England), July 1995, 43(1), pp. 37-42

[13] Feix, M.; Wolf, L.; Schweikert, H. U.

Distribution of 17beta-Hydroxy Steroid Dehydrogenases in Human Osteoblast-like Cells.

Mol Cell Endocrinol (Ireland), Jan. 22, 2001, 171(1-2), pp. 163-4

[14] Jensen, E. V.; Jacobson, H. I.

Basic Guides to the Mechanism of Estrogen Action.

Recent Progress in Hormone Research, 1962, 18, pp. 387-414

[15] Kuiper, G. G.; Gustafsson, J. A.

The Novel Estrogen Receptor-beta Subtype: Potential Role in the Cell- and Promoter-specific Actions of Estrogens and Anti-estrogens.

FEBS Lett (Netherlands), Jun. 23, 1997, 410(1), pp. 87-90

[16] Gustafsson, J. A.

Therapeutic Potential of Selective Estrogen Receptor Modulators.

Curr Opin Chem Biol (England), August 1998, 2(4), pp. 508-11

[17] Korach, K. S.

Estrogen Receptor Knock-Out Mice: Molecular and Endocrine Phenotypes

J Soc Gynecol Investig (United States), January-February 2000, 7(1 Suppl), pp. S16-7

[18] Temple, J. L.; Fugger, H. N.; Li, X., et al.

Estrogen Receptor Beta Regulates Sexually Dimorphic Neural Responses to Estradiol.

Endocrinology (United States), January 2001, 142(1), pp. 510-3

[19] Weihua, Z.; Saji, S.; Makinen, S., et al.

Estrogen Receptor (ER) Beta, A Modulator of ER Alpha in the Uterus.

Proc Natl Acad Sci USA (United States), May 23, 2000, 97(11), pp. 5936-41

[20] Dourakis, S. P.; Tolis, G.

Sex Hormonal Preparations and the Liver.

Eur J Contracept Reprod Health Care (England), March 1998, 3(1), pp. 7-16

[21] von Schoultz, B., Carlstrom, K.; Collste, L., et al.

Estrogen Therapy and Liver Function-Metabolic Effects of Oral and Parenteral Administration.

Prostate (United States), 1989, 14(4), pp. 389-95

[22] Tikkanen, M. J.

The Menopause and Hormone Replacement Therapy: Lipids, Lipoproteins, Coagulation and Fibrinolytic Factors.

Maturitas (Ireland), March 1996, 23(2), pp. 209-16

[23] Schunkert, H.; Danser, A. H.; Hense, H. W., et al.

Effects of Estrogen Replacement Therapy on the Renin-Angiotensin System in Postmenopausal Women.

Circulation (United States), Jan. 7, 1997, 95(1), pp. 39-45

[24] Gambreli, R. D.

Strategies to Reduce the Incidence of Endometrial Cancer in Postmenopausal Women.

Am J Obstet Gynecol (United States), November 1997, 177(5), pp. 1196-204;

Discussion 1204-7

[25] von Schoultz, B.; Soderqvist, G.; Cline, M., et al.

Hormonal Regulation of the Normal Breast.

Maturitas (Ireland), May 1996, 23 Suppl, pp. S23-5

[26] Soderqvist, G.

Effects of Sex Steroids on Proliferation in Normal Mammary Tissue.

Ann Med (England), December 1998, 30(6), pp. 511-24

[27] Cummings, S. R.; Eckert, S.; Krueger, K. A., et al.

The Effect of Raloxifene on Risk of Breast Cancer in Postmenopausal Women: Results from the MORE Randomized Trial. Multiple Outcomes of Raloxifene Evaluation.

JAMA (United States), Jun. 16, 1999, 281(23), pp. 2189-97

[28] Bush, T. L.; Blumenthal, R.; Lobo, R. et al.

SERMs and Cardiovascular Disease in Women. How Do These Agents Affect Risk?

Postgrad Med (United States), March 2001, Spec No, pp. 17-24

[29] Kuiper, G. G., Carisson, B.; Grandien, K., et al.

Comparison of the Ligand Binding Specificity and Transcript Tissue Distribution of Estrogen Receptors Alpha and Beta.

Endocrinology (United States), March 1997, 138(3), pp. 863-70

[30] Jung-Testas, I.; Groyer, M. T.; Bruner-Lorand, J., et al.

Androgen and Estrogen Receptors in Rat Ventral Prostate Epithelium and Stroma.

Endocrinology (United States), October 1981, 109(4), pp. 1287-9

[31] Stack, G.; Gorski, J.

Relationship of Estrogen Receptors and Protein Synthesis to the Mitogenic Effect of Estrogens.

Endocrinology (United States), November 1985, 117(5), pp. 2024-32

[32] Fuhrmann, U.; Slater, E. P.; Fritzemeier, K. H.

Characterization of the Novel Progestin Gestodene by Receptor Binding Studies and Transactivation Assays.

Contraception (United States), January 1995, 51(1), pp. 45-52

1. Use of estrogen receptor (ER)β-selective agonists for the production of a pharmaceutical agent for triggering somatotropic and/or organotropic effects in the CNS, the circulatory system, the skeletal system and/or the immune system in the aging male and female organism (anticatabolic therapy).

2. Use according to claim 1 for stimulation of the growth hormone.

3. Use according to claim 1 for stimulation of IGF-I.

4. Use according to claim 1 for influencing (increasing) the growth of muscle mass.

5. Use according to claim 1 for preserving muscle mass.

6. Use according to claim 1 for influencing (increasing) the growth of bone mass.

7. Use according to claim 1 for preserving bone mass.

8. Use according to claim 1 for influencing the growth of the thymus.

9. Use according to claim 1 for preserving the thymus and its function.

10. Use according to claim 1 for increasing the IGF-I level.

11. Use according to claim 1 for stimulation of the adrenal secretion of androgenic hormones (DHEA, DHEA-S, androstenedione).

12. Use according to claim 1 for stimulation of testicular hormone secretion (testosterone).

13. Use according to claim 1 for increasing the level of HDL-cholesterol.

14. Use according to claim 1 for improving the state of nutrition.

15. Use of an estrogen receptor (ER)β-selective agonist according to claim 1 in a dosage of 10 μg to 10 mg daily absolute.

16. Use of 8β-vinyl-1,3,10-estratriene-3,17β-diol according to claim 1.
